(19) United States
(12) Patent Application Publication
(10) Pub. No.: US 2014/0287446 A1
(43) Pub. Date: Sep. 25, 2014

(54) Monoclonal Antibody For Acetylamantadine

(71) Applicant: BioMark Technologies Inc., Vancouver (CA)

(72) Inventors: Brian Cheng, Chesterfield, MO (US); Rashid Bux, Vancouver (CA); Gregorio Aversa, Vancouver (CA); Bram Ramjiawan, Winnipeg (CA); Daniel Sitar, Winnipeg (CA)

(73) Assignee: BioMark Technologies Inc., Vancouver (CA)

(21) Appl. No.: 14/116,743

(22) PCT Filed: May 11, 2012
(86) PCT No.: PCT/CA12/50307

§ 371 Date: Mar. 13, 2014

Related U.S. Application Data
(60) Provisional application No. 61/484,521, filed on May 10, 2011.

(51) Int. Cl. C12Q 1/48; C07K 16/44

(52) U.S. Cl. CPC C12Q 1/48; C07K 16/44
USPC 435/15; 530/388.9; 435/70.1; 530/391.7

(57) ABSTRACT

A method of producing an antibody comprises immunizing a mammal with an amine-derivative of acetylamantadine, immunizing the mammal with acetylamantadine, and producing the antibody from the mammal. The antibody recognizes acetylamantadine but does not recognize amantadine.

### CROSS-REFERENCE TO RELATED APPLICATION

This application claims priority from U.S. Provisional Patent Application Ser. No. 61/484,521 which was filed on May 10, 2012 and the full disclosure of which is incorporated herein by reference.

### BACKGROUND OF THE INVENTION

1. Field of the Invention

The present invention relates to methods and compositions for the quantification of spermine/spermidine N¹-acetyltransferase (SSAT) enzymatic activity.

2. Description of the Related Art

Spermidine/spermine N¹-acetyltransferase (SSAT), is ubiquitously distributed in mammalian tissues and plays a role in catabolism and elimination of polyamines from cells. SSAT is an inducible enzyme that catalyzes the transfer of an acetyl group from an acetyl-coenzyme A to the aminopropyl moiety of the polyamines. This action by SSAT facilitates polyamine degradation, excretion, and cycling and/or intracellular cycling. In this manner SSAT participates in the maintenance of polyamine homeostasis in mammalian cells. However, in normal or un-induced mammalian tissues SSAT is present at very low levels.

Induction of SSAT expression can be caused by different drugs, growth factors, polyamines, polyamine analogues, toxic substances, hormones and physiological stimuli. Although all of the aforementioned compounds could cause induction of SSAT expression, induction occurs at different times for each individual compound. The regulation of SSAT expression occurs at the levels of transcription, mRNA stability, mRNA translation and protein stability. Induction or over-expression of SSAT is usually required for there to be sufficient SSAT enzyme present in cells or 100,000×g supernatant before in-vitro experiments can be successfully undertaken.

While current literature teaches that SSAT is an acetylating enzyme specifically for substrates including spermine and spermidine or its analogues, SSAT activity, SSAT enzyme kinetics and assay methodology for non-spermine/spermidine substrates of SSAT has not been understood. Current methods exist to quantify SSAT activity. However these techniques are dependent on highly skilled personnel and complicated experimental methods. More specifically, there has been a need for assay methodology which quantifies the activity of SSAT through detection of acetylated forms of non-spermine. Spermidine substrates of SSAT, including amantadine may be used to detect various pathological conditions.

Traditional methods such as Gas Liquid Chromatography (GLC), High Pressure Liquid Chromatography (HPLC) alone or coupled with mass spectroscopy are being used for assaying acetylated metabolite such as acetylamantadine. The detection sensitivity requires parts per billion of the target analyte which becomes a challenge. GLC and HPLC have been shown to be effective for in-vitro assays but may not be practical for in-vivo assays. Employment of deuterated analyte as an internal standard for HPLC-MS-MS method to assay acetylamantadine was successful.

These traditional methods require labor intensive analytical service resulting in high operating cost and capital cost. All these add to the inefficiency of the healthcare economy. In addition, biological samples must be logistically handled and shipped to a centralized laboratory for assay. These operations may result in sample quality changes and result in false interpretation of the result.

A test methodology at the point of care is therefore desirable to minimize sample instability and reduce healthcare cost. The form of in-vitro testing diagnostic (IVD) at the clinical office will also allow quick medical decisions.

### SUMMARY OF THE INVENTION

It is an object of the present invention to provide an antibody, or functional equivalents or functional parts thereof, which recognizes acetylamantadine but does not recognize amantadine.

There is accordingly provided a method of producing an antibody comprising immunizing a mammal with an amine-derivative of acetylamantadine, immunizing the mammal with acetylamantadine, and producing the antibody from the mammal. The method may include conjugating the amine-derivative of acetylamantadine to an ovalbumin carrier with 1-ethyl-3-(3-dimethylaminopropyl) carbodiimide and emulsifying the acetylamantadine-ovalbumin conjugate in Freund's adjuvant. The method may also include immunizing the mammal with avidin coupled to the acetylamantadine. The animal may be boosted with the amine-derivative of acetylamantadine. The antibody may be prepared by obtaining a sample of peripheral blood monocytic cells from the mammal, culturing the peripheral blood monocytic cells under conditions where the B lymphocytes are polyclonally activated, and activating the B lymphocytes to proliferate and differentiate into antibody-secreting cells. The antibody produced may be a monoclonal antibody.

The monoclonal antibody may be specific for acetylamantadine generated from antibody-producing cells from a mammal immunized with acetylamantadine conjugated to a carrier protein together with an adjuvant.

The monoclonal antibody may be specific for acetylamantadine which was prepared by intramuscular immunization of a mammal with 10-500 μg of the acetylamantadine-ovalbumin conjugate emulsified in complete Freund's Adjuvant.

The monoclonal antibody may be specific for acetylamantadine which was prepared by intramuscular immunization of a mammal with 10-500 μg of the acetylamantadine-ovalbumin conjugate emulsified in complete adjuvant.

The monoclonal antibody may be specific for acetylamantadine which was prepared by intramuscular immunization of a mammal with 100-400 μg of the acetylamantadine-ovalbumin conjugate emulsified in complete Freund's Adjuvant.

The monoclonal antibody may be specific for acetylamantadine which was prepared by intramuscular immunization of a mammal with 200-300 μg of the acetylamantadine-ovalbumin conjugate emulsified in complete Freund's Adjuvant.

The monoclonal antibody may be specific for acetylamantadine which was prepared by intramuscular immunization boost of a mammal 2-15 times every 1-6 weeks with acetylamantadine-ovalbumin conjugate 10-200 μg given with alum as an adjuvant.

The monoclonal antibody may be specific for acetylamantadine which was prepared by intramuscular immunization boost of a mammal 2-15 times every 1-6 weeks with acetylamantadine-ovalbumin conjugate 20-150 μg given with alum as an adjuvant.

The monoclonal antibody may be specific for acetylamantadine which was prepared by intramuscular immunization boost of a mammal 2-15 times every 1-6 weeks with acetylamantadine-ovalbumin conjugate 30-100 μg given with alum as an adjuvant.

The monoclonal antibody may be specific for acetylamantadine which was prepared by intramuscular immunization boost of a mammal 2-15 times every 2-5 weeks with acetylamantadine-ovalbumin conjugate 10-200 μg given with alum as an adjuvant.

The monoclonal antibody may be specific for acetylamantadine which was prepared by intramuscular immunization boost of a mammal 2-15 times every 2-5 weeks with acetylamantadine-ovalbumin conjugate 20-150 μg given with alum as an adjuvant.

The monoclonal antibody may be specific for acetylamantadine which was prepared by intramuscular immunization boost of a mammal 2-15 times every 2-5 weeks with acetylamantadine-ovalbumin conjugate 30-100 μg given with alum as an adjuvant.

The monoclonal antibody may be specific for acetylamantadine in which the mammals boosted by intramuscular immunization four weeks after the last boost, were immunized with 1-8 μg of avidin bound with an excess of biotinylated acetylamantadine with alum as an adjuvant.

The monoclonal antibody may be specific for acetylamantadine in which the mammals were boosted by intramuscular immunization four weeks after the last boost, were immunized with 2-6 μg of avidin bound with an excess of biotinylated acetylamantadine with alum as an adjuvant.

The monoclonal antibody may be specific for acetylamantadine in which the mammals were boosted by intramuscular immunization four weeks after the last boost, were immunized with 3-5 μg of avidin bound with an excess of biotinylated acetylamantadine with alum as an adjuvant.

The monoclonal antibody may include any functionally equivalent antibody or functional parts thereof, which the antibody discriminates between acetylamantadine and amantadine and thus detects enzymatic activity.

The monoclonal antibody may be used as a quantification tool for acetylamantadine.

The monoclonal antibody may be used as a quantification tool for the detection of spermine/spermidine N¹-acetyltransferase activity.

The monoclonal antibody may be used as a diagnostic tool to determine spermine/spermidine N¹-acetyltransferase in a patient.

### DESCRIPTIONS OF THE PREFERRED EMBODIMENTS

Disclosed herein is an example of a successful immunization of animals, rabbits in this example, according to the following improved protocol. This enabled the demonstration that individual antibodies could be raised that could discriminate between acetylamantadine and amantadine. This showed that monoclonal antibodies could be raised that specifically bind to acetylamantadine but these same antibodies did not bind to amantadine.

### Preparation of Acetylamantadine Protein Conjugates

Rabbits were immunized with a conjugate of a carrier protein ovalbumin conjugated by 1-ethyl-3-(3-dimethylaminopropyl) carbodiimide to an amine-derivative of acetylamantadine using the manufacturer's instructions. In this example, the manufacturer was Thermo Fisher Scientific Inc. of 3747 North Meridian Road, Rockford, Ill., United States of America, 61101.

The amine-derivative of acetylamantadine was synthesized as described in the following synthetic scheme.

### Synthesis of Adamantylamine Derivatives

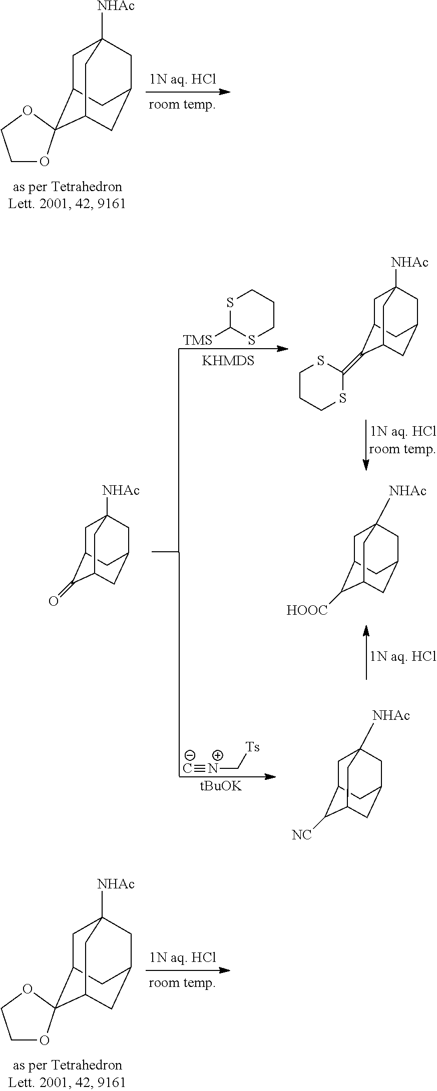

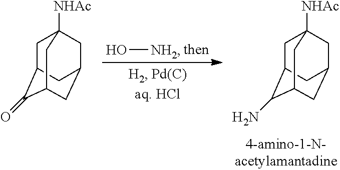

A conjugate of the biotin-binding protein avidin was coupled to an excess of biotinylated 4-amino-1-N-acetylamantadine. The biotinylation was carried out using standard methods using instructions from the manufacturer which, in this example, was Thermo Fisher Scientific Inc. of 3747 N Meridian Rd, Rockford, Ill., United States of America, 61101.

### Immunization of Rabbits

Two New Zealand white rabbits were immunized intramuscularly with 250 ug of the acetylamantadine-ovalbumin conjugate emulsified in complete Freund's Adjuvant. They were boosted by intramuscular injections given seven times every four weeks with acetylamantadine-ovalbumin conjugate (30-100 ug) and with alum as an adjuvant. Four weeks after the last boost the rabbits were immunized with 4 μg of avidin coupled with an excess of acetylamantadine with alum as an adjuvant. Five weeks later 20 ml of peripheral blood monocytic cells were collected from an ear vein.

### Preparation of Peripheral Blood Monocytic Cells

The peripheral blood monocytic cells (PBMC) were separated by Ficoll Hypaque. Then the PBMC were cultured in micro-cultures in 96-well plates at limiting dilution at one of ten thousand PBMC per culture, five thousand PBMC, or one thousand PBMC per well, plating 72 cultures at each concentration of APBMC per well. The PBMC were cultured under conditions where the B lymphocytes were polyclonally activated by the inclusion of helper T lymphocytes and a mixture of cytokines from activated T lymphocytes, for example, as disclosed in Zubler, R. H. F. Erard, et al. (1985). Mutant EL-4 thymoma cells polyclonally active murine and human B cells via direct interaction. Journal of Immunology. 134(6): 3662-8, the full disclosure of which is incorporated herein by reference. In these conditions about one in three B lymphocytes were randomly activated to proliferate or clone and differentiate into antibody-secreting cells. After a week, the supernatants of all of the micro-cultures were harvested and then tested by ELISA for the presence of antibodies that bound acetylamantadine.

### Assay of Supernatants of Limit Dilution Culture for Acetylamantadine-Specific Antibodies

ELISA plates were coated with the biotin-binding protein Streptavidin and blocked with skim milk. An excess of biotinylated acetylamantadine was added to the Streptavidin-coated ELISA plates so that acetylamantadine was bound to the Streptavidin and then washed of excess biotinylated acetylamantadine. 50 ul of the various supernatants were added to the wells of the ELISA assay, and left overnight for antibodies to bind. The ELISA plates were then washed with phosphate-buffered saline with an automated washer. Then enzyme-conjugated goat anti-rabbit antibodies were added to bind to any rabbit antibodies bound to the acetylamantadine. The substrate was added and the ELISA developed by standard methods and the optical densities were assayed by an ELISA reader. The results are shown below in Tables 1 and 2.

TABLE 1
Table 1 ELISA assay of reactivity of antibodies in the supernatants
with acetylamantadine. As described, ELISA plates coated with
acetylamantadine were used to assay 72 supernatants from micro-
cultures in which 5000 PBMC had been cultured as described. Shown
are the optical density readings. Note that 11 out of 72 supernatants
had readings above background. Thus these 11 supernatants contained
rabbit antibodies that bound acetylamantadine.

| Group: | | | | | |
|---|---|---|---|---|---|
| Sample | Wells | Values | Wells | Values | MeanValue |
| AcetylAman | B1 | 0.023 | E1 | 0.026 | 0.158 |
| | B2 | 2.054 | E2 | 0.011 | |
| | B3 | 0.068 | E3 | 0.013 | |
| | B4 | 0.025 | E4 | 0.004 | |
| | B5 | 0.021 | E5 | 0.026 | |
| | B6 | 0.033 | E6 | 0.027 | |
| | B7 | 0.044 | E7 | 0.103 | |
| | B8 | 0.029 | E8 | 1.162 | |
| | B9 | 0.088 | E9 | 1.232 | |
| | B10 | 0.011 | E10 | 0.014 | |
| | B11 | 0.01 | E11 | 0.01 | |
| | B12 | 0.016 | E12 | 0.024 | |
| | C1 | 0.206 | F1 | 0.02 | |
| | C2 | 0.022 | F2 | 0.054 | |
| | C3 | 0.01 | F3 | 0.028 | |
| | C4 | 0.023 | F4 | 0.048 | |
| | C5 | 0.045 | F5 | 0.014 | |
| | C6 | 0.02 | F6 | 2.006 | |
| | C7 | 0.006 | F7 | 0.171 | |
| | C8 | 0.02 | F8 | 0.018 | |
| | C9 | 0.008 | F9 | 0.676 | |
| | C10 | 0.04 | F10 | 0.009 | |
| | C11 | 0.271 | F11 | 0.016 | |
| | C12 | 0.09 | F12 | 0.048 | |
| | D1 | 0.425 | G1 | 0.02 | |
| | D2 | 0.02 | G2 | 0.013 | |
| | D3 | 0.06 | G3 | 0.682 | |
| | D4 | 0.021 | G4 | 0.032 | |
| | D5 | 0.088 | G5 | 0.016 | |
| | D6 | 0.032 | G6 | 0.015 | |
| | D7 | 0.004 | G7 | 0.064 | |
| | D8 | 0.016 | G8 | 0.015 | |
| | D9 | 0.408 | G9 | 0.042 | |
| | D10 | 0.022 | G10 | 0.007 | |
| | D11 | 0.036 | G11 | 0.01 | |
| | D12 | 0.059 | G12 | 0.311 | |

TABLE 2
Table 2 ELISA assay of reactivity of antibodies in the supernatants
with amantadine: demonstration that rabbit antibodies could
discriminate between acetylamantadine and amantadine. As described,
ELISA plates coated with acetylamantadine were used to re-
assay the 72 supernatants from Table 1 (from micro-cultures
in which 5000 PBMC had been cultured). Shown are the optical
density readings. Note that 8 out of the 11 supernatants that
contained rabbit antibodies that bound acetylamantadine, had
background readings and thus did not contain antibodies that
reacted with amantadine. Thus these 8 supernatants contained
antibodies that statistically were likely to be monoclonal
and that discriminated between acetylamantadine and amantadine.
Note that three of the 11 supernatants that contained rabbit
antibodies that bound acetylamantadine, also cross-reacted
with amantadine.

| Group: | | | | | |
|---|---|---|---|---|---|
| Sample | Wells | Values | Wells | Values | MeanValue |
| Amantadine HCl | B1 | 0.002 | E1 | 0.013 | 0.024 |
| | B2 | 0.009 | E2 | 0.017 | |
| | B3 | 0.007 | E3 | 0.008 | |
| | B4 | 0.01 | E4 | 0.003 | |
| | B5 | 0.015 | E5 | 0.014 | |
| | B6 | 0.023 | E6 | 0.047 | |
| | B7 | 0.123 | E7 | 0.024 | |
| | B8 | 0.016 | E8 | 0.011 | |
| | B9 | 0.019 | E9 | 0.007 | |
| | B10 | 0.01 | E10 | 0.001 | |
| | B11 | 0.006 | E11 | 0.008 | |
| | B12 | 0.02 | E12 | 0.019 | |
| | C1 | 0.047 | F1 | 0.011 | |
| | C2 | 0.017 | F2 | 0.021 | |
| | C3 | 0.006 | F3 | 0.014 | |
| | C4 | 0.013 | F4 | 0.025 | |
| | C5 | 0.071 | F5 | 0.008 | |
| | C6 | 0.011 | F6 | 0.018 | |
| | C7 | 0.004 | F7 | 0.004 | |
| | C8 | 0.009 | F8 | 0.006 | |
| | C9 | 0.016 | F9 | 0.005 | |
| | C10 | 0.011 | F10 | −0.001 | |
| | C11 | 0.09 | F11 | 0.005 | |
| | C12 | 0.026 | F12 | 0.009 | |
| | Dl | 0.128 | G1 | 0 | |
| | D2 | 0.014 | G2 | 0.011 | |
| | D3 | 0.027 | G3 | 0.298 | |
| | D4 | 0.01 | G4 | 0.032 | |
| | D5 | 0.035 | G5 | 0.015 | |
| | D6 | 0.006 | G6 | 0.027 | |
| | D7 | 0.017 | G7 | 0.116 | |
| | D8 | 0.005 | G8 | 0.014 | |
| | D9 | 0.009 | G9 | 0.01 | |
| | D10 | 0.008 | G10 | 0.005 | |
| | D11 | 0.023 | G11 | 0.011 | |
| | D12 | 0.025 | G12 | 0.001 | |

### Analysis of the Results

It was observed that at a concentration of 5,000 PBMC per culture, eleven out of seventy two (or 15%) of the micro-cultures supernatants tested positive for antibodies that bound acetylamantadine. It was mathematically likely that each of these wells contained a single clone of B lymphocytes that produced antibodies that bound acetylamantadine.

### Antibody Specificity

The specificity of these antibodies for acetylamantadine was then tested to determine whether any of the antibodies could discriminate between acetylamantadine and amantadine using an ELISA assay for antibodies that could bind amantadine. To do this, 4-amino-1-N-amantadine was biotinylated using standard methods. The ELISA plates were coated with Streptavidin and blocked and an excess of biotinylated amantadine was added to the ELISA plates. After allowing the amantadine to bind to the Streptavidin, the ELISA plates were washed and 50 μl aliquot of each of the supernatants was added. The ELISA was developed using standard methods and determined the proportion of the monoclonal antibodies that bound acetylamantadine was specific for acetylamantadine and did not bind amantadine, and thus would be useful for detecting the acetyl modification of amantadine. The frequency of antibodies that only bound amantadine was also determined.

As can be seen from Table 3, eleven of the seventy two supernatants from the 5,000 PBMC per well bound to acetylamantadine and, of these, eight only bound acetylamantadine and thus could discriminate between acetylamantadine and amantadine.

TABLE 3
Table 3 Seventy two microcultures were set up with each
containing 5,000 PBMC from a mixture of blood from two
rabbits immunized with acetylamantadine as described. After
seven days the supernantant from each well was sampled
and aliquots were assayed for antibodies against acetylamantadine
or amantadine as described. Shown are results from the
eleven wells out of seventy two in which the optical density
(OD) in the ELISA for antibodies against acetylamantadine
was above the background. Also shown are the results on
the same wells of ELISA against amantadine. The eight wells
that contained antibodies which discriminated between acetylamantadine
and amantadine are shown in bold. The other three wells
had reactivity with both acetylamantadine and amantadine.
There were another three wells (not shown) that had antibodies
that reacted with amantadine but not acetylamantadine.

| | OD | OD |
|---|---|---|
| Micro-culture | acetylamantadine | amantadine |
| B2 | 2.054 | 0.009 |
| C1 | 0.206 | 0.047 |
| C11 | 0.271 | 0.090 |
| D1 | 0.425 | 0.128 |
| D9 | 0.408 | 0.009 |
| E8 | 1.162 | 0.011 |
| E9 | 1.232 | 0.007 |
| F6 | 2.006 | 0.018 |
| F9 | 0.676 | 0.005 |
| G3 | 0.682 | 0.296 |
| G12 | 0.311 | 0.001 |

This demonstrated that monoclonal antibodies existed in this rabbit that discriminated between acetylamantadine and amantadine. The frequency was at least 8 per (72×5,000) PBMC which means that it was 8 per 360,000 PBMC. As there were about a million PBMC per ml and there were ˜200 mls of blood in a rabbit, this meant that one rabbit contained about 5000 B lymphocytes that made antibodies that could bind acetylamantadine but did not bind amantadine.

The present invention accordingly provides a novel method and composition comprising highly specific and highly effective antibodies having the ability to have specific recognition of acetylamantadine and to highly discriminate the specific parent molecule amantadine. These antibodies are particularly useful to quantify acetylamantadine. The quantification of acetylamantadine can be used to quantify SSAT activity and elevated SSAT activity is an indication of diseases including, but not limited, to inflammations and cancers.

It will be understood by a person skilled in the art that there are many methods that could be used to produce monoclonal antibodies from animals immunized in a similar manner including, but not limited to, hybridoma techniques and cell-immortalization techniques.

It will further be understood by a person skilled in the art that there are many ways that these antibodies could be raised. Other animals including, but not limited to, mammals such as mice, rats, hamsters, sheep or goats may be immunized. Other carrier proteins such as keyhole limpet hemocyanin may be used and other methods of cross linking the acetylamantadine to the carrier protein may also be used.

It will still further be understood by a person skilled in the art that many of the details provided above are by way of example only, and are not intended to limit the scope of the invention which is to be determined with reference to the following claims.

1. An antibody which recognizes acetylamantadine but does not recognize amantadine.

2. The antibody as claimed in claim 1 wherein the antibody is a monoclonal antibody.

3. A method of producing an antibody which recognizes acetylamantadine but does not recognize amantadine, the method comprising:
immunizing a mammal with an amine-derivative of acetylamantadine;
immunizing the mammal with acetylamantadine; and
producing the antibody from the mammal.

4. The method as claimed in claim 3 further including conjugating the amine-derivative of acetylamantadine to an ovalbumin carrier.

5. The method as claimed in claim 4 further including conjugating the amine-derivative of acetylamantadine to the ovalbumin carrier with 1-ethyl-3-(3-dimethylaminopropyl) carbodiimide.

6. The method as claimed in claim 4 further including emulsifying the amine- derivative of acetylamantadine and the ovalbumin carrier in Freund's adjuvant.

7. The method as claimed in claim 3 further including immunizing the mammal with avidin coupled to. the acetylamantadine.

8. The method as claimed in claim 3 further including boosting the mammal with the amine-derivative of acetylamantadine.

9. The method as claimed in claim 3 further including:
obtaining a sample of peripheral blood monocytic cells from the mammal;
culturing the peripheral blood monocytic cells under conditions where the B lymphocytes axe polyclonally activated; and
activating the B lymphocytes to proliferate and differentiate into antibody-secreting cells.

10. Use of the antibody as claimed in claim 1 as a diagnostic tool to determine spermine/spermidine N′-acetyltransferase in a patient.
